(19) United States
(12) Patent Application Publication
(10) Pub. No.: US 2012/0003177 A1
(43) Pub. Date: Jan. 5, 2012

(54) CURCUMIN-CONTAINING POLYMERS AND WATER-SOLUBLE CURCUMIN DERIVATIVES AS PRODRUGS OF PRODRUG CARRIERS

(76) Inventors: Youqing Shen, Laramie, WY (US); Huadong Tang, Laramie, WY (US); Edward Van Kirk, Laramie, WY (US); William Murdoch, Laramie, WY (US); Maciej Radosz, Laramie, WY (US)

(21) Appl. No.: 13/119,454

(22) PCT Filed: Sep. 17, 2009
(86) PCT No.: PCT/US09/57303

§ 371 Date: May 11, 2011

Related U.S. Application Data
(60) Provisional application No. 61/097,671, filed on Sep. 17, 2008.

(51) Int. Cl. A61K 31/775; C08F 212/14; C07C 49/205; A61K 31/12; A61P 35/00; A61K 31/704; A61K 38/14; A61K 33/24; A61K 31/337; C08G 8/26; A61K 31/437

(52) U.S. Cl. 424/78.31; 528/127; 526/313; 424/78.38; 424/78.37; 568/325; 514/679; 514/283; 514/34; 514/19.2; 424/649; 514/449; 514/281

(57) ABSTRACT

Curcumin, a polyphenol extracted from the rhizome turmeric, has been polymerized to produce a polymer material having a backbone of one or more repeating structural units, at least one of which comprises a curcumin monomer residue. These curcumin-containing polymers have a wide range of pharmacological activities, including, among others antitumor, antioxidant, antiinflammatory, antithrombotic and antibacterial activities. Certain species of these polymers have exhibited remarkable antitumor activity. Water-soluble curcumin derivatives and their use as prodrugs and prodrug carriers are also disclosed.

### CROSS-REFERENCE TO RELATED APPLICATIONS

The present application claims the benefit of U.S. Provisional Patent Application No. 61/097,671, filed Sep. 17, 2008, the entire disclosure of which is incorporated by reference herein.

### STATEMENT REGARDING FEDERAL SPONSORED RESEARCH OR DEVELOPMENT

The present invention was made with funds provided by the National Institute of Health.

### BACKGROUND OF THE INVENTION

Curcumin (1,7-bis-(4-hydroxy-3-methoxyphenyl)-hepta-1,6-diene-3,5-dione) is a hydrophobic polyphenol isolable from the rootstock of perennial Curcuma longa. Curcumin has long been used as a food spice, and as a traditional herb for wound healing and treatment of liver disease in ancient India and China. Curcumin was found to have a wide range of biological and pharmacological activities, such as antioxidant,¹ antiinflammatory,^{2,3} antithrombotic,^{4,5} antidiabetes,^{6,7} antibacterial,^{8,9} antihepatotoxic,^{10,11} antiarthritic,^{12,13} antirheumatoid,¹⁴ and anti-Alzheimer's disease activities.¹⁵⁻¹⁷ Curcumin was also reported to inhibit HIV-I integrase protein, decrease total cholesterol and LDL cholesterol level, but increase the beneficial HDL cholesterol level in serum.^{18,19}

Recently, this natural product has attracted considerable interest due to its antitumor and tumor prevention properties. As an antioxidant, curcumin shows strong antiproliferative effects and thus is considered a potential cancer therapy reagent. It has been reported that curcumin interacts with multiple cellular targets, such as nuclear factor-kappa B (NF-κB) and transcription factor activator protein-1 (AP-1), and binds more than 30 proteins.^{20,22} Curcumin inhibits various interleukins and multiple protein kinase (e.g., PKC, JNK), and suppress the expression of human epidermal growth factor receptor (HER-2), epidermal growth factor receptor (EGFR), and estrogen receptor (ER).^{21,23,24} Curcumin was also found to down-regulate multidrug resistance proteins (MDR) and P-glycoprotein (P-gp) and has the potential to overcome cancer cell multidrug resistance.^{25,26} In vitro, curcumin demonstrated cytotoxicity against a wide variety of cancer cells lines such as DU145 prostate carcinoma, A549 lung carcinoma, and HT29 colon carcinoma with an IC₅₀ (50% inhibitory concentration) of about 10˜75 μM^{27,28} In vivo, curcumin to showed preventive and therapeutic effects against human tumors such as pancreas, breast, ovarian, ascites, colorectal and brain carcinomas.^{29,30}

Curcumin has been proved pharmacologically safe even at very high doses in many clinical studies and various animal models. For example, curcumin showed no toxicity at a daily oral dose as high as 12 g in a phase I clinical trial and no dose-limiting toxicity was found for curcumin in another phase II trial.^{31,32} However, in spite of its demonstrated pharmacological safety and wide efficacies in a variety of human diseases, curcumin has not been approved as a drug or therapeutic agent, due at least, in part, to its low adsorption, rapid metabolism and limited bioavailability.³³ Curcumin is strongly hydrophobic, making it practically insoluble in water at acidic conditions, and is rapidly degraded at neutral and alkaline conditions. For example, curcumin has a half life (t_{1/2}) time less than 10 min in PBS at pH 7.2.³⁴ In patients administrated curcumin at a dose of 0.45-3.6 g/day, only a few nanomoles of curcumin were detected in the patient's peripheral or portal circulation.³² In another study, curcumin was found to have no appreciable inhibitory activity with respect to lung and breast tumors because of its low bioavailability.³⁵ The aqueous insolubility and poor bioavailability is regarded as a major impediment to successful clinical utilization of curcumin.

Thus, curcumin has been loaded in liposomes,^{29,36} hydrogels,³⁷ polymer blends,³⁸ solid dispersions,^{39,40} nanoparticles,^{41,42} and conjugated to dendrimer⁴³ and other carriers to improve its stability and bioavailability. Safavy et al. reported that curcumin conjugated to a polyethylene glycol (PEG) chain with an ester linker was inactive. Although the conjugate using a liable urethane linkage showed higher cytotoxicity than the pristine curcumin against PC-3 pancreatic carcinoma cells, it was not stable and readily hydrolyzed at neutral conditions (pH 7.4, t_{1/2}=60 min or 200 min depending on PEG chain length).⁴⁴ Other disadvantages, such as insufficient loading capacity, low loading efficiency, thermal and storage instability, burst release of the drug in the blood stream and rapid clearance by reticuloendothelial system, have been observed for these previously described drug loading systems.^{29,42}

Prior experience with curcumin-polymer conjugates is fairly typical of polymer-drug conjugates in general, when the drug molecule is chemically linked to the end(s) of the polymer backbone, or incorporated as part of a pendent side chain on the polymer backbone. Conjugates prepared in this way often exhibit low drug loading efficiency and insufficient drug content in the polymer, since the polymer usually has much higher molecular weight that small molecule drugs. Thus, novel curcumin-containing polymer materials, having improved solubility and bioavailability, are needed to allow the full therapeutic benefit of curcumin to be realized.

### SUMMARY OF THE INVENTION

In accordance with one aspect of the present invention, there is provided a copolymer comprising curcumin as a constituent monomer.

In one embodiment, the polymer has a backbone of repeating structural units, which may be the same or different, and at least one of which comprises a curcumin monomer residue.

In a further embodiment, the curcumin monomer residue is chemically bound to at least one other monomer residue such that the repeating structural unit comprises a polyester.

In another embodiment, the curcumin monomer residue is chemically bound to at least one other monomer residue, such that the repeating structural unit comprises a polyether.

In still another embodiment, the repeating structural units constituting the polymer are different, with the polymer backbone also including a repeating structural unit that comprises a polyether glycol residue.

According to another aspect, the present invention provides a curcumin derivative of the formula:

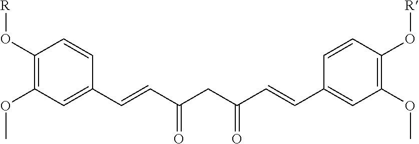

wherein one or both of R and R′ represent a water-soluble moiety, and when only one of R and R′ represent a water-soluble moiety, the other represents hydrogen.

In yet another aspect of the invention, colloidal particles are provided, which comprise either the curcumin-containing polymers or the curcumin derivatives described herein. The colloidal particles may be in the form of vesicles or nanoparticles. These colloidal particles are readily adapted for use in pharmaceutical preparations and can act as prodrugs for the delivery of curcumin, or prodrug carriers for other therapeutic agents, e.g., anti-neoplastic agents.

According to still another aspect of this invention, there is provided a method for the treatment of cell proliferative diseases, especially cancers, which comprises administration of the aforementioned pharmaceutical preparations to a patient in need of such treatment. The method of the invention may also be applied to the treatment of inflammatory disorders.

The symmetric structure and bi-hydroxyl functionality of the curcumin molecule is used to advantage by directly incorporating curcumin in at least one of the repeating structural units of the polymers of this invention in which curcumin forms part of the polymer backbone. These high molecular weight curcumin-containing polymers have many desirable properties, including high curcumin-loading content and efficiency, excellent thermal and storage stability, easy molecular weight control, and adjustable hydrolysis kinetics. It is expected that such polymers have broader pharmaceutical applications, in addition to their use as antitumor agents, including such uses as antioxidants, antiinflammatory, antithrombotic, antifungal and antibacterial agents.

### BRIEF DESCRIPTION OF THE DRAWINGS

FIG. 1 is a graphical representation of the dose response curves resulting from cytotoxicity testing of water-soluble curcumin-containing polymers of the present invention, which also included curcumin, per se, as a control.

FIG. 2 is a graphical representation of the dose response curves obtained from cytotoxicity testing of a curcumin-containing polyether-polyethylene glycol copolymer of the present invention in cancer cell lines in addition to SKOV-3.

FIG. 3 is a graphical representation of the results of an evaluation of the in vivo antitumor activity of a curcumin-containing polyether-polyethylene glycol copolymer of the present invention using SKOV-3 xenografts.

FIG. 4 shows graphical representations of colloidal particle size distribution, by volume, for certain curcumin-containing vesicles made using PEG (molecular weight of 187) (FIG. 4a); and, by intensity, for certain curcumin-containing nanoparticles made using PEG (molecular weight of 454) (FIG. 4b).

FIG. 5 shows the results of cytotoxicity assays in which SKOV-3 ovarian cancer cells were treated with varying amounts of curcumin, using as prodrugs a curcumin-containing polymer of the invention (FIG. 5a), and a curcumin derivative of the invention (FIG. 5b).

FIG. 6 shows the results of a cytotoxicity assay in which SKOV-3 ovarian cancer cells were treated with varying amounts of curcumin, using one of the curcumin derivatives described herein as a prodrug vesicle carrier for another antineoplastic agent, namely, camptothecin (CPT), in an amount of 5 wt % of CPT based on the curcumin derivative. The vesicles, which contained curcumin derivatives at a dose of 1.0 μg/ml (a), 0.5 μg/ml (b), 0.1 μg/ml (c), 0.05 μg/ml (d), were compared to 1.0 μg/ml curcumin prodrug only (e) and a control (f).

FIG. 7 shows the results of a cytotoxicity assay in which KM12 colon cancer cells were treated with varying amount of curcumin, using a curcumin derivative of the invention as a prodrug.

### DETAILED DESCRIPTION OF THE INVENTION

Curcumin may be isolated from the root of Curcuma longa according to procedures known in the art⁵⁴. Synthetic routes for the production of curcumin have also been described⁵⁵.

The curcumin-containing polymers of the invention have repeating structural units of the following structural formula:

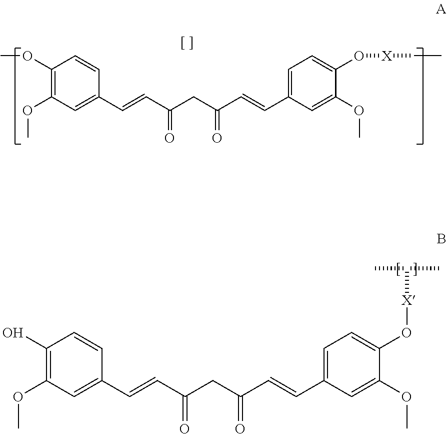

wherein the X moiety represents any biologically compatible comonomer residue capable of chemically binding to the hydroxy groups of curcumin; and X′ represents any suitable linker that connects curcumin to a carrier polymer.

The curcumin-containing polymers of formula A may be formed as homopolymers, or as copolymers with additional repeating structural units, which may comprise curcumin linked to a different X moiety or which are free of curcumin, with polyether and polyester copolymer repeating units being preferred.

The term “the same”, when used in reference to repeating structural units of the curcumin-containing polymers of the invention, means substantially the same, but not necessarily identical. The term thus allows for chemical structure variation(s) that normally occur in polymerization reactions of the type described herein.

The term “monomer residue”, as used herein, refers to the molecular structure of a monomer present in the resulting polymer after polymerization is complete, e.g., due to the separation of water, hydrochloric acid or the like in a polycondensation reaction.

Representative examples of suitable co-monomers include, without limitation, polycarboxylic acids, including, e.g., di-, tri-, tetra- and penta-carboxylic acids, polycarboxylic acyl halides, polycarboxylic acid anhydrides, divinyl compounds, dihalide compounds, and polyetherglycols. Representative examples of useful polycarboxylic acids and anhydrides include, without limitation, oxalic acid, succinic acid, 3,3′-dithiodipropronic acid, terephthalic acid, benzophenone-3,3′,4,4′-tetracarboxylic dianhydride, cyclobutane-1,2,3,4-tetracarboxylic dianhydride, tetrahydrofuran-2,3,45-tetracarboxylic anhydride, diethylene-triaminepentacetic acid anhydride, pyromellitic dianhydride.

Representative examples of useful divinyl compounds include, without limitation, divinyl sulfone, divinyl sulfoxide, 1,4-butanediol divinyl ether, 1,4-cyclohexanedimethanol divinyl ether, 1,4-divinyl-1,1,2,2,3,3,4,4-octamethyltetrasilane, bis[4-(vinyloxy)butyl]adipate, tri(ethylene glycol) divinyl ether, di(ethylene glycol) divinyl ether and polyethylene glycol divinyl ether.

Representative examples of polycarboxylic acyl halides include, without limitation, oxalyl chloride, malonyl chloride, succinyl chloride, glutaric acid dichloride, phthaloyl dichloride. Suitable dihalide compounds include, without limitation, ethyl dichlorophosphate, phosphinic dichloride, phosphonic dichloride, platinum(H)diammine dichloride, dichlorosilane, allyl(dichloro)methylsilane, dichloro-cyclohexyl-methylsilane, dichloro(methyl)phenylsilane, dichloro-methyl-octadecylsilane.

Representative examples of useful polyetherglycols include, without limitation, polyethyleneglycol (PEG), polypropyleneglycol (PPG), and polyethyleneglycol-polypropylene glycol copolymers.

The constituent repeating structural units may be selected to enhance the water-solubility of the resulting polymer. The polyetherglycol compounds, such as PEG and PPG, are useful for this purpose, and may be incorporated into the polymer backbone, or grafted as a side chain to the polymer backbone. Other examples of such moieties include, without limitation, polyvinylpyrrolidone (PVP), polyvinyl alcohol (PVA), polyethyleneimine (PEI) and poly[N-(2-hydroxypropyl)methacrylamide](PHPMA), polyglutamic acid.

The polyetherglycol, when used, is added to the polymerization reaction medium in an amount such that the content of the polyetherglcyol residue in the resulting copolymer is between about 2 and 70 weight percent of the copolymer. The polyethylene glycols used in the practice of this invention preferably have a is molecular weight in the range of 200-20000.

The carrier polymers can be water soluble polymers such as polyglutamic acid, PEG, PVP, PVA, PEI, and PHPMA. The linker(s) (X′) can be comonomer residues, such as dicarboxylic acids, polycarboxylic acids, polycarboxylic acid anhydrides, polycarboxylic acyl halides, divinyl compounds, dihalide compounds or the like.

The curcumin-containing polymers of the present invention typically have a number-average molecular weight in the range of about 500 to about 500,000, and preferably 10,000 to 100,000.

The curcumin-containing polymers having the repeating structural unit of formula A, above, can be prepared, as exemplified below, by polycondensation of curcumin either with a polycarboxylic anhydride, polycarboxylic acid, or divinyl compound to form the desired product. All polycurcumins were designed in a manner such that they are stable at neutral physiological conditions but hydrolysable under acidic conditions and degradable in cancer tissues. In the polyester homopolymers and copolymers prepared as exemplified below, the ester bond was stable at physiological pH (7.4) but hydrolyzable at lower pH (5˜6). The polycurcumins having disulfide bonds are sensitive to the concentration of glutathione, a thiol-containing tripeptide that can reduce and beak disulfide bond. Since the glutathione concentration is very low in blood (in micromolar range) but sufficiently high (in milimolar range) in cancer tissues to cause the scission of disulfide bond,⁴⁵ such polymers can remain stable in blood circulation but quickly degrade and release curcumin in cancer tissues to kill cancer cells. The ether bonds in the polyether homopolymers and copolymers were found to be stable at neutral and basic conditions, but hydrolyzable in acidic conditions. Unlike normal tissues, solid tumor tissues usually have an acidic extracellular environment and an altered pH gradient across their cell compartments.^{46,47} For example, the pH of tumor extracellular fluid has been measured to be 6.81±0.09 on average with lowest value of 5.55.⁴⁸ The polycurcumins of the invention, therefore, would be expected to be stable in normal tissue intercellular fluid and in blood, but readily hydrolyzed and degraded in acidic extracellular fluid of cancer tissues to release curcumin for therapeutic action, which can increase the polycurcumins blood circulation time, lower their side effect to normal tissues and improve therapeutic efficacy in cancer tissues.

Because each repeating unit of the polycurcumin homopolymers described herein incorporates one curcumin monomer, the curcumin content of these polymers is very high, as will be discussed below. However, due to the strong hydrophobicity of curcumin, if the loading content of curcumin is too high the polymer is rendered water-insoluble. Therefore, short PEG chains are beneficially incorporated into the polycurcumins to adjust their water solubility. Longer PEG chains impact better water-solubility, but lower the curcumin loading content. The polycurcumin properties can thus be readily controlled and tuned by modifying the PEG chain length, or the feeding ratio between curcumin and PEG. Depending on different applications, the curcumin loading content can vary from several percent to sixty or seventy percent.

The polycurcumin-containing polymers of this invention can be easily polymerized to high molecular weight. Table 2 lists the molecular weights of representative examples of polycurcumins, as measured, using a Waters gel permeation chromatograph equipped with two 300 mm Waters Styrgel solvent-saving columns (molecular weight ranges: 5×10²-3×10⁴, 5×10³-6×10⁵), a Waters 2414 refractive index detector, and a Precision 1102 laser-light scattering detector. The eluent was THF at a flow rate of 0.3 mL/min with column temperature of 30 ° C. A series of polystyrene standards were used to calibrate the light scattering detector. Gel permeation chromatography (GPC) data were recorded and processed using a Waters software package.

The theoretical loading content in Table 2 was calculated from the feeding ratio between curcumin and other polymer components, such as 3,3′-dithiodipropionic acid and PEG, using the following equation:

$$Drug loading content ( \% ) = \frac{\begin{matrix}amount of curcumin \\ incorporated into polycurcumin\end{matrix}}{weight of the polycurcumin} \times 100 \%$$

The measured loading content was determined by ¹H NMR based on the integrations of curcumin aromatic protons and the specific protons of other polymer components, such as ethylene protons in PEG. The molecular weights of the curcumin-containing polymers ranged from 10⁴ to 10⁵, with polydispersities between 1.4 and 2.6, which is typical for polycondensation reactions. The high molecular weights of curcumin-containing polymers suggested that the condensation reactions had a high polymerization degree and a high monomer conversion (>95%), indicating almost all curcumin and comonomers were incorporated into the polymer product and nearly no curcumin remained after polymerization. Thus, the loading content can be calculated according to the feeding ratio of curcumin and other polymer components. In other words, the measured loading content should be close to the calculated theoretic loading content, which has been confirmed in Table 2. The negligible difference between theoretical and measured loading content was caused either by the integration error in ¹H NMR or small constitutional changes during the precipitation and purification process of polycurcumins. Unlike other drug loading systems, such as micelle and liposomal carriers, the drug loading content in curcumin-containing polymer described herein, in which curcumin is incorporated into the polymer backbone can be accurately and easily controlled and designed by changing the curcumin feeding ratio.

The drug loading efficiency was calculated using the following equation:

$$Drug loading efficiency ( \% ) = \frac{\begin{matrix}amount of curcumin \\ incorporated into polycurucmin\end{matrix}}{amount of curcumin intially added} \times 100 \%$$

For the same reason stated above, the loading efficiency in the curcumin-containing polymers described herein should be close to 100%, since all curcumin monomers were incorporated into the backbones of the resulting polymers, as shown in Table 1.

TABLE 1
Molecular weight, solubility, curcumin loading
content and efficiency of polycurcumins

| | | | Solu- | | | |
|---|---|---|---|---|---|---|
| | | | bility | Theoretical | Measured | Loading |
| | Mn | | in | loading | Loading | effi- |
| Entry | (×10⁴) | PDI | water* | content | Content | ciency |
| Example 1 | 2.95 | 2.4 | S | 21.2% | 20% | 94% |
| Example 2 | 1.38 | 2.6 | S | 17.1% | 18% | 100% |
| Example 3 | 1.15 | 2.1 | S | 22.0% | 20% | 91% |
| Example 4 | 6.13 | 1.5 | I | 67.9% | 66% | 97% |
| Example 5 | 1.73 | 1.9 | S | 13.4% | 14% | 100% |
| Example 6 | 1.28 | 1.8 | PS | 36.0% | 34% | 94% |
| Example 7 | 2.14 | 2.2 | I | 64.3% | 64% | 99% |
| Example 8 | 4.50 | 1.4 | S | 23.8% | 21% | 88% |

*S: Soluble; PS: Partially soluble; I: Insoluble

The curcumin derivatives of the invention can be prepared simply by chemical modification with suitable reactants that are effective to make them water-soluble. Representative examples of such reagents include, without limitation, tetraethyleneglycol methyl vinyl ether, polyethyleneglycol, polyethyleneimine, polyvinyl alcohol, and polyglutamic acid.

Suitable reagents can also impart an amphiphilic character to curcumin derivatives so as to form colloidal particles. The average particle size of colloidal particles composed of the curcumin-containing copolymers or oligomers and the curcumin derivatives described herein is in the range of about 10 to about 1,000 nanometers in diameter. These colloidal particles can be prepared with supplemental therapeutic agents incorporated therein, and thereby function as prodrug carriers for the supplemental agents. For example, curcumin-based vesicles may be loaded with one or more anti-neoplastic agents, such as camptothecin, doxorubicin, cis-platin, paclitaxel, bleomycin, aclarubicin, chromomycin, peplomycin, vincristin, colchioinamide, curcumin and etc

Specific examples of the preparation of curcumin-containing copolymers and curcumin derivatives in accordance with the present invention are provided below.

Insofar as is known, animal studies to date have not conclusively established to an LD₅₀ for free curcumin administration. Oral doses of free curcumin as high as 500 mg/kg and intravenous doses of 40 mg/kg have been tolerated in rats⁵⁶. Useful dosages of the pharmaceutical preparations of this invention can be determined by comparing their in vitro activity and the in vivo activity in animal models. Methods for extrapolation of effective dosages in mice, and other test animals, to humans are known in the art⁵⁷.

Methods for determining whether a given dosage is effective for treating a specific form of cancer are well known in the art and include, for example, assessment based on a decrease in the number of malignant cells (i.e., a decrease in cell proliferation or a decrease in tumor size). As will be understood by those skilled in the art, the method of treatment of the present invention may produce a lasting and complete response, or a partial or transient clinical response⁵⁸.

Assays to test for malignant cell death are also well known in the art and include, for example, standard dose response assays that assess cell viability; agarose gel electrophoresis of DNA extractions of flow cytometry to determine DNA fragmentation, as a characteristic of cell death; assays that measure the activity of polypeptides involved in apoptosis; and assays for morphological signs of cell death. Other assays include chromatin assays (i.e., counting the frequency of condensed nuclear chromatin) or drug resistance assays^{59,60}.

The method of the present invention can be applied for the treatment of pathological conditions, for example, arising out of excessive proliferation of cells in a patient in need thereof, which includes mammals, preferably humans. The term “treatment”, as used herein, refers to the therapeutic, prophylactic or inhibitory treatment of such conditions. The cell proliferative diseases which may be treated using the method of this invention include, without limitation, cancer and autoimmune disease. The method can be used to treat breast cancer, ovarian cancer, non-small cell lung cancer, small cell lung cancer, squamous cell cancer of the head and neck, malignant melanomas, pancreatic cancer, and other type cancers. Examples of the autoimmune diseases that may be treated using the method of this invention include, without limitation, systemic lupus eythematous, multiple sclerosis and psoriatic arthritis.

The method described herein may also be utilized for the treatment of inflammatory conditions or disorders including, for example, asthma or rheumatoid arthritis.

The curcumin-containing copolymers and curcumin derivatives described herein, whether formulated as prodrugs or prodrug carriers for other therapeutic agents, may be administered using any route of administration effective for the treatment of the aforementioned diseases or disorders. Administration may be is carried out by intraperitoneal injection, intravenous injection subcutaneous injection, oral administration, or via the gastrointestinal tract, with administration dosage depending on the disease.

The following examples describe the invention in further detail. These examples are provided for illustrative purposes only and should in no way be construed as limiting the invention.

Examples 1-8 describe general procedures for the synthesis of representative curcumin-containing polymers, both homopolymers and copolymers, within the scope of this invention. The repeating structural units of the curcumin-containing polymers prepared in Examples 1-8 are shown in Table 2.

TABLE 2

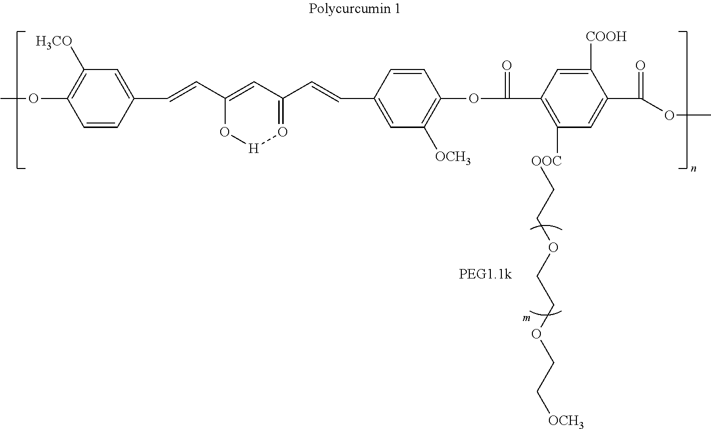

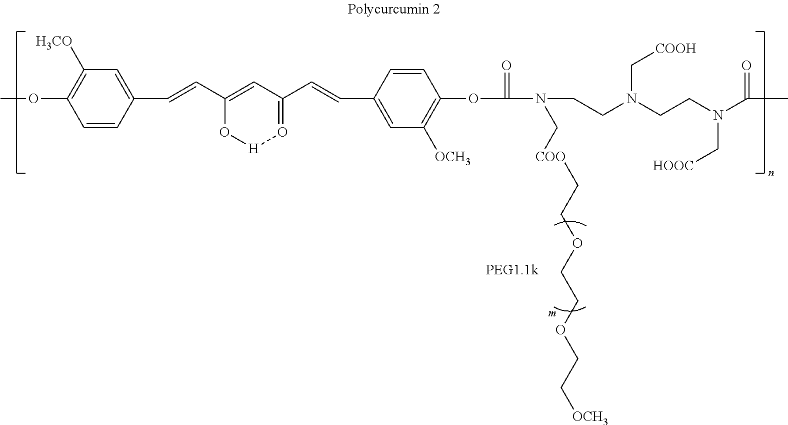

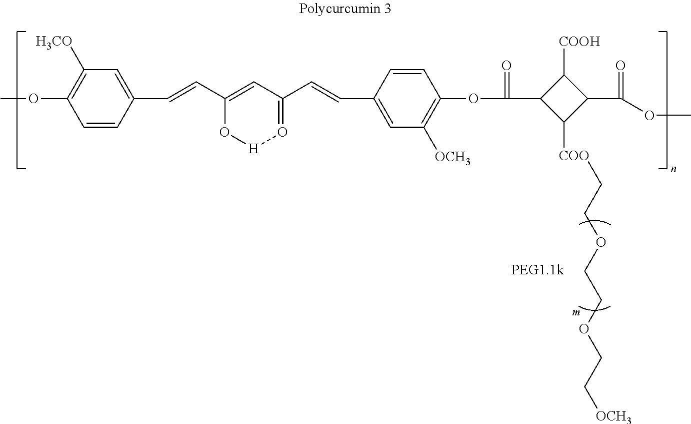

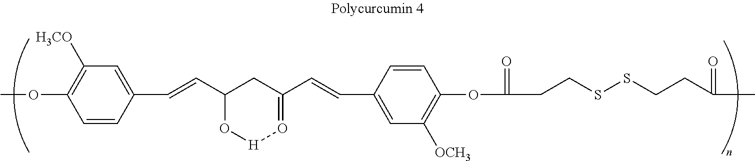

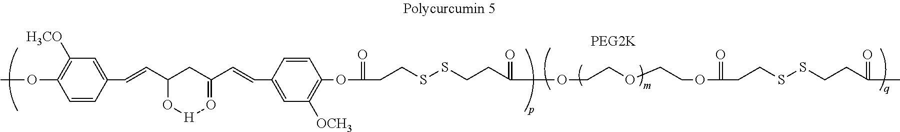

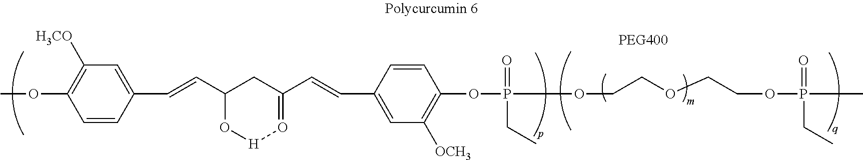

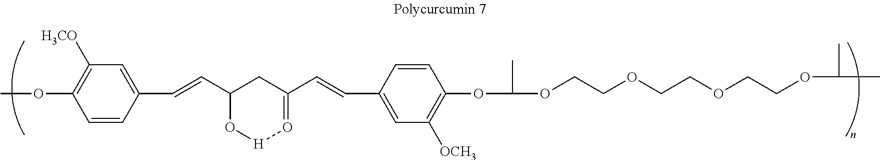

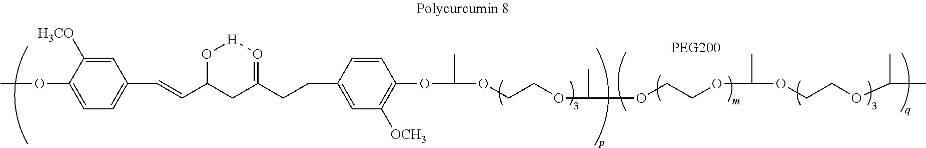

m, n, p, and q are the numbers of repeating unit

In carrying out these syntheses, the following materials were used:

Tri(ethylene glycol) divinyl ether (98%), trifluoroacetic acid (99%), toluene 4-sulfonic acid (TSA 98%), cyclobutane-1,2,3,4-tetracarboxylic dianhydride (99%), N,N′-dicyclohexylcarbodiimide (DCC, 99%) 3,3′-dithiodipropionic acid, pyromellitic dianhydride (99%), 4-dimethylaminopyridine (DMAP, 99%), triethylamine (99%), polyethylene glycol (PEG) monomethyl ether (Mn=1.1 k), 3-mercaptopropionic acid (99%), ethyl dichlorophosphate (98%) and diethylenetriaminepentaacetic dianhydride (99%), all purchased from Aldrich and used as received; Poly(ethylene glycol) (PEG, Mn=200, Mn=400, Aldrich, 99%), dried over calcium hydride. Curcumin (high purity, Axxora LLC), further purified by repeated recrystallization in methanol.

¹H NMR spectra were recorded on a Bruker Advance DRX-400 spectrometer. Deuterated acetone (acetone-d₆) or chloroform (CDCl₃) was dried over molecular sieve overnight before use. Chemical shift δ was given in ppm referenced to the internal standard tetramethylsilane (TMS, δ=0 ppm).

### Example 1

### Polyester Having a Repeating Unit Comprising Curcumin and Pyromellitic Anhydride Monomer Residues and a Polyetheylene Glycol (PEG) Monomethyl Ether Side-Chain Bound to the Polymer Backbone

2.000 g of curcumin and 1.183 g of pyromellitic dianhydride were dissolved in 50 mL anhydrous dimethylsulfoxide (DMSO). After the mixture was stirred at 50° C. for 24 hours, an excess of anhydrous tetrahydrofuran (THF) was added to precipitate the polymer product. This product was washed with anhydrous THF and then 1.00 g of this product was redissolved in 40 mL anhydrous DMSO, followed by addition of 2.00 g of polyethylene glycol monomethyl ether (Mn=1.1 k), 0.40 g of N,N′-dicyclohexylcarbodiimide (DCC), and 0.1 g of 4-dimethylaminopyridine (DMAP). This solution was stirred at room temperature for 24 hours and an excess of anhydrous ether was then added to precipitate the final product, which was further purified by reprecipitation from THF with anhydrous ether and dried under vacuum to produce 2.2 g (yield 75%) of deep yellow soft solid polymer. ¹H NMR (acetone-d₆, δ, ppm): 8.4 (br, 2H, C₆H₂(COO)₄), 7.6 (d, 2H, CH_{b}═CH_{c}), 6.9˜7.3 (br, m, 6.1H, C₆H₃), 6.7 (d, 2H, CH_{b}═CH_{c}), 6.0 (s, 1H, CH_{d}═C—OH), 4.2 (br, 2.1H, COOCH₂CH₂O), 3.9 (s, 6H, CH₃OC₆H₃), 3.5˜3.7 (br, 118H, OCH₂CH₂O), 3.4 (s, 3.4H, CH₂CH₂OCH₃). Based on the integration of curcumin aromatic protons and PEG ethylene protons, the curcumin loading content was calculated to be 20% and each unit was averagely conjugated with 1.2 PEG chain.

### Example 2

### Polyester Having a Repeating Unit Comprising Curcumin and Diethylenetriamine Pentaacetic Dianhydric Monomer and a Peg Monomethyl Ether Side Chain Bound to the Polymer Backbone

The procedure for synthesis of polycurcumin 2 is the same as that for polycurcumin 1 except using 1.940 g of diethylenetriaminepentaacetic dianhydride to replace 1.183 g of pyromellitic dianhydride. Yield: 75%. ¹H NMR (acetone-d₆, δ, ppm): 7.6 (d, 2H, CH_{b}═CH_{c}), 6.9˜7.3 (m, 6.1H, C₆H₃), 6.7 (d, 2H, CH_{b}═CH_{c}), 6.0 (s, 1H, CH_{d}═C—OH), 4.2 (br, 2.1H, COOCH₂CH₂O), 3.9 (s, 6H, —CH₃OC₆H₃), 3.5˜3.7 (br, 128H, OCH₂CH₂O), 3.4 (s, 3.4H, CH₂CH₂OCH₃), 2.6˜2.7 (m, 8H, NCH₂CH₂N). Based on the integration of curcumin aromatic protons and PEG ethylene protons, the curcumin loading content was calculated to be 18% and each unit was averagely conjugated with 1.3 PEG chain.

### Example 3

### Polyester Having a Repeating Unit Comprising Curcumin and Cyclobutane-1,2,3,4-Tetracarboxylic Dianhydride Monomer Residues and a Polyethylene Glycol (PEG) Monomethyl Ether Side-Chain Bound to the Polymer Backbone

The procedure for synthesis of polycurcumin 3 is the same as that for polycurcumin 1 except using 1.065 g of cyclobutane-1,2,3,4-tetracarboxylic dianhydride to replace 1.183 g of pyromellitic dianhydride. Yield: 70%. ¹H NMR (CDCl₃, δ, ppm): 7.6 (br, 2H, CH_{b}═CH_{c}), 6.9˜7.2 (m, 6H, C₆H₃), 6.6 (br, 2H, CH_{b}═CH_{c}), 5.9 (br, 1H, CH_{d}═C—OH), 4.3 (br, 2H, COOCH₂CH₂O), 3.9 (s, 6H, CH₃OC₆H₃), 3.5˜3.7 (br, 112H, OCH₂CH₂O), 3.4 (s, 3.7H, CH₂CH₂OCH₃). Based on the integration of curcumin aromatic protons and PEG ethylene protons, the curcumin loading content was calculated to be 20% and each unit was averagely conjugated with 1.1 PEG chain.

### Example 4

### Polyester Having a Repeating Unit Comprising Curcumin and 3,3′-Dithiodipropionic Acid Residues

1.000 g of curcumin, 0.571 g of 3,3′-dithiodipropionic acid and 1.15 g DCC and 0.1 g of DMAP were dissolved in 40 mL anhydrous THF. After the mixture was stirred at room temperature for one day, an excess of cold anhydrous ether was added to precipitate the polymer and the polymer was further purified by reprecipitation from THF with cold anhydrous ether and dried under vacuum at room temperature to get 1.2 g (yield 81%) deep yellow solid polycurcumin 4. ¹H NMR (CDCl₃, δ, ppm): 7.6 (d, 2H, CH_{b}═CH_{c}), 6.9˜7.2 (m, 6H, C₆H₃), 6.6 (d, 2H, CH_{b}═CH_{c}) 5.9 (s, 1H, CH_{d}═C—OH), 3.90 (s, 6H, CH₃OC₆H₃), 2.9˜3.2 (m, 8.4H, —CH₂CH₂—S—S—CH₂CH₂—). Based on the integration of curcumin aromatic protons and the ethylene protons in 3,3′-dithiodipropionic acid, the curcumin loading content was determined to be 66%.

### Example 5

### Polyester Copolymer Having a Repeating Unit Comprising Curcumin and 3,3′-Dithiodipropionic Acid Monomer Residues, and a Repeating Unit Comprising PEG and 3,3′-Dithiodipropionic Acid Monomer Residues

0.876 g of curcumin, 1.000 g g of 3,3′-dithiodipropionic acid, 4.755 g PEG (Mn=2 k) and 2.0 g DCC and 0.1 g of DMAP were dissolved in 80 mL anhydrous THF. After the mixture was stirred at room temperature for one day, an excess of anhydrous ether was added to precipitate the polycurcumin 5 and the polymer was further purified by reprecipitation from THF with anhydrous ether and dried under vacuum at room temperature to get 5.2 g (yield 80%) yellow powder. ¹H NMR (CDCl₃, δ, ppm): 7.6 (br, 2H, CH_{b}═CH_{c}), 6.9˜7.2 (br, 6H, C₆H₃), 6.6 (br, 2H, CH_{b}═CH_{c}) 5.8 (br, 1H, CH_{d}═C—OH), 4.2 (s, 2H, COOCH₂CH₂O), 3.90 (s, 6H, CH₃OC₆H₃), 3.5˜3.7 (br, 173H, OCH₂CH₂O), 2.9˜3.2 (m, 17H, —CH₂CH₂—S—S—CH₂CH₂—). Based on the integration of curcumin aromatic protons, ethylene glycol protons, and ethylene protons in 3,3′-dithiodipropionic acid, the curcumin loading content was determined to be 14%.

### Example 6

### Polyester Copolymer Having a Repeating Unit Comprising Curcumin and Ethyl Dichlorophosphate Monomer Residues, and a Repeating Unit Comprising PEG and Ethyl Dichlorophosphate Monomer Residues

0.736 g of curcumin, 0.8 g PEG (Mn=400), 0.652 g of ethyl dichlorophosphate, and 1.1 mL of triethylamine were dissolved in 40 mL anhydrous THF. The solution was stirred at 50° C. for 12 hours, and then the THF was evaporated under vacuum and 40 mL of chloroform was added to dissolve the polymer. After the chloroform solution was washed with distilled water to remove triethylamine salt, an excess of ether was added to precipitate the polycurcumin 6 and the polymer was further purified by reprecipitation from THF with excess of cold anhydrous ether and dried under vacuum at room temperature to give 1.8 g (yield 87%) yellow soft solid. ¹H NMR (CDCl₃, δ, ppm): 7.6 (br, 2H, CH_{b}═CH_{c}), 6.0˜7.2 (br, 9H, C₆H₃, CH_{b}═CH_{c} and CH_{d}═C—OH), 4.3 (br, 4.2H, POOCH₂CH₂O), 3.5˜3.9 (br, 38H, OCH₂CH₂O), 1.3 (br, 7H, P—CH₂CH₃). Based on the integration of curcumin aromatic protons, ethylene glycol protons, and ethyl protons in phosphate, the curcumin loading content was determined to be 34%.

### Example 7

### Polyether Having a Repeating Unit Comprising Curcumin and Triethylene Glycol Divinyl Ether Monomer Residues

1.800 g of curcumin, 1.000 g of tri(ethylene glycol) divinyl ether, and 10 μg of toluene 4-sulfonic acid were dissolved in 40 mL anhydrous THF. After the solution was stirred at 50° C. overnight, hexane was added to precipitate the polycurcumin 7, which was further purified by reprecipitation from THF with hexane and dried under vacuum at room temperature to get 2.4 g (yield 86%) yellow solid. ¹H NMR (acetone-d₆, δ, ppm): 7.6 (br, 2H, CH_{b}═CH_{c}), 6.8˜7.4 (br, 8H, C₆H₃ & CH_{b}═CH_{c}), 4.7 (br, 2.1H, (CH₃)CH), 3.9 (br, 6H, CH₃OC₆H₃), 3.4˜3.7 (br, 13H, OCH₂CH₂O), 1.2 (br, 6.714, (CH₃)CH). Based on the integration of curcumin aromatic protons and the ethylene glycol protons, the loading content was calculated to be 64%.

### Example 8

### Polyether Copolymer Having a Repeating Unit Comprising Curcumin and Triethylene Glycol Divinyl Ether Monomer Residues and a Repeating Unit Comprising PEG and Triethylene Glycol Divinyl Ether Monomer Residues

Synthesis of polycurcumin 8. 1.10 g curcumin (3.0 mmol), 1.40 g polyethylene glycol 200 (7.0 mmol), 2.12 g tri(ethylene glycol) divinyl ether (10.5 mmol), and 20 μg toluene 4-sulfonic acid were dissolved in 50 mL anhydrous tetrahydrofuran. After the solution was stirred at 50° C. overnight, an excess of cold anhydrous ether was added to precipitate the conjugate, which was further purified by reprecipitation from THF with cold anhydrous ether to give 3.6 g (yield 78%) of yellow soft solid polycurcumin 8. ¹H NMR (CDCl₃, δ, ppm): 7.3˜7.7 (br, 2H, CH_{b}═CH_{c}), 6.3˜7.2 (br, 8H, C₆H₃ & CH_{b}═CH_{c}), 4.80 (q, 6.2H, —(CH₃)CH—), 3.5˜3.8 (br, 97H, OCH₂CH₂O), 1.8 (d, 20H, —(CH₃)CH—) Based on the integration of curcumin aromatic protons and PEG ethylene protons, the curcumin loading content was calculated to be 21%.

The following two examples describe the results of biological activity testing of a number of curcumin-containing polymers of this invention.

The materials used in conducting these biological activity experiments included:

Permount, purchased from Sigma-Aldrich (St Louis, Mo.); 4′,6-diamidino-2-phenylindole, dihydrochloride (DAPI); purchased from Invitrogen Corporation (Carlsbad, Calif.); BrdU cell proliferation kit purchased from Thermo Fisher Scientific Inc. (Waltham, Mass.); Primary antibodies purchased from Santa Cruz Biotechnology, Inc. (Santa Cruz, Calif.); and Secondary antibodies purchased from Jackson ImmunoResearch Laboratories, Inc. (West Grove, Pa.).

The human ovarian carcinoma SKOV-3 cell line was purchased from American type culture collection (ATCC). Cells were cultured in RPMI 1640 medium supplemented with 10% fetal bovine serum (HyClone, Logan, Utah) and grown at 37° C. in a humidified atmosphere of 5% CO2 (v/v) in air. All of the experiments involving cells were performed on cells in the exponential growth phase.

### Example 9

### In Vitro Cytotoxicity of Polycurcumins to SKOV-3 Cancer Cell Lines

According to the American Cancer Society, ovarian cancer is among the fifth most common cancer and the fifth most common cause of cancer death in women. The SKOV-3 ovarian cancer cell line of human origin was f used initially to screen the cytotoxicity of the polycurcumins of this invention.

The cytotoxicity of polycurcumins was determined using the standard 3-(4,5-dimethylthiazolyl-2)-2,5-diphenyltetrazolium bromide (MTT) cell proliferation kit (ATCC, Manassas, Va.) according to manufacturer's protocol. In brief, SKOV-3 cells were seeded onto 96-well plates with a density of 15,000 cells per well and incubated at 37° C. in a humidified atmosphere of 95% air and 5% CO₂ for 16 h. The medium in each well was replaced with 200 μL of culture medium containing the treatments and cultured for 72 h. The medium in each well was then replaced with fresh media and the cells were incubated for another 24 h. The incubation medium was then replaced with 100 μL of fresh medium and 10 μL of MIT reagent. After 6 h, 100 μL of detergent reagent was added to each well and incubated for 18 h at room temperature in the dark until all the crystals dissolved. The absorbance intensity at 570 nm was recorded on a Bio-Rad (model 550) microplate reader. Cell viability is defined as the percent live cells compared with untreated controls.

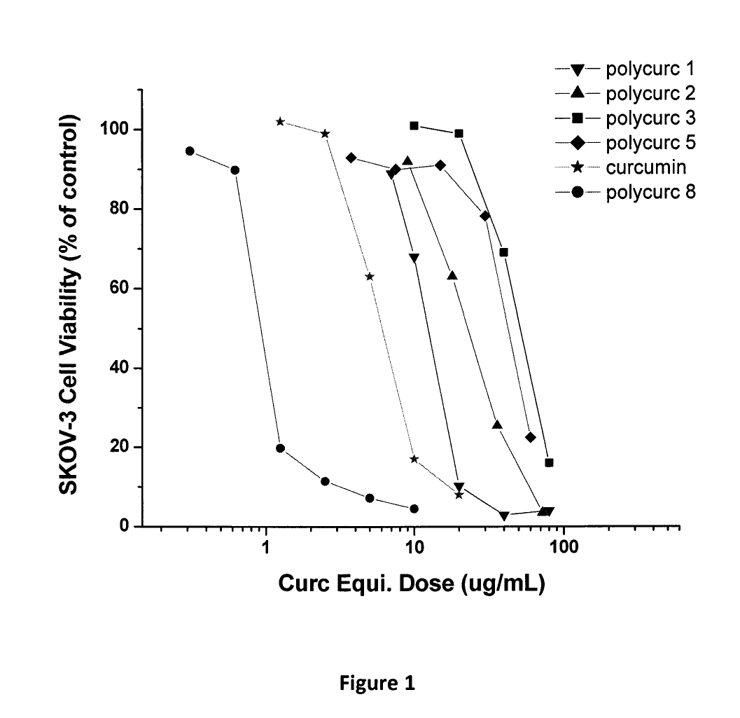

The cytotoxicities of the polycurcumins prepared as described in Examples 1, 2, 3, 5 and 8, above, were evaluated in this experiment. The polycurcumins of Examples 4, 6 and 7 were not tested due to their poor solubilities in water. Curcumin, per se, used as a control, was tested by dissolving curcumin in DMSO at a concentration of 5 mg/mL, followed by dilution in a cell-containing medium to the required doses. MTT assay results are shown in FIG. 1. Based on the dose response curve, the IC₅₀ (50% cell inhibitory concentration) of each polycurcumin was determined and the results are summarized in Table 3. The IC₅₀ in terms of the polycurcumin dose (IC_{50-P}) and that in terms of curcumin-equivalent dose (IC_{50-C})) is exchangeable according to the equation: IC_{50-C}=IC_{50-P}×loading content (LC). For each polycurcumin tested, the commoners such as 3,3′-dithiodipropionic acid, pyromellitic dianhydride, tri(ethylene glycol) divinyl ether and PEG, were used as controls. These controls showed no significant cytotoxicity against SKOV-3 cells, which means that the cytotoxicity of polycurcumins is attributable solely to the curcumin present therein.

TABLE 3
IC₅₀ of polycurcumins to SKOV-3 human ovarian cancer cell line

| | Ex. 1 | Ex. 2 | Ex. 3 | Ex. 5 | Ex. 8 | Curcumin |
|---|---|---|---|---|---|---|
| Entry | (μg/mL) | (μg/mL) | (μg/mL) | (μg/mL) | (μg/mL) | (μg/mL) |
| IC_{50-P} | 63.5 | 123.8 | 277.0 | 301.3 | 5.7 | / |
| L.C. | 20% | 18% | 20% | 15% | 21% | / |
| IC_{50-C} | 12.7 | 22.3 | 55.4 | 45.2 | 1.2 | 7.8 |

Table 3 shows that the polycurcumins of Examples 2, 3 and 5 had lower cytotoxicity to SKOV-3 cells, while the polycurcumin of Example 1 had cytotoxicity comparable to curcumin, per se. The polycurcumin of Example 8 had the highest cytotoxicity with much lower IC₅₀ (1.2 μg/mL) than that of curcumin.

### Example 10

### Cytotoxicity of the Polycurcumin of Example 8 to MCF-7 and OVCAR Cancer Cell Lines

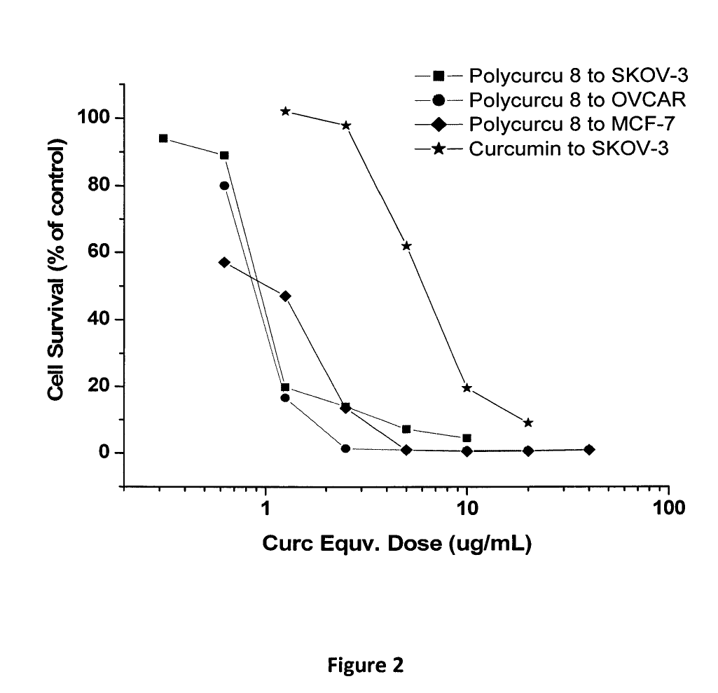

The cytotoxicity of the polycurcumin of Example 8 to other cancer cell lines, namely, MCF-7 breast carcinoma and OVCAR carcinoma, was also assessed using standard MTT assay. FIG. 2 shows the dose response curves of the polycurcumin of Example 8 to OVCAR and MCF-7 cells. As a basis of comparison, the curves for curcumin and the polycurcumin of Example 8 with respect to SKOV-3 were also included. As is evident from FIG. 2, the polycurcumin of Example 8 was not only highly cytotoxic to SKOV cells, but also had strong cytotoxicities to OVCAR and MCF-7. The IC₅₀ was determined to be 1.2 μg/mL (3.3 μM), 1.4 μg/mL (3.8 μM) and 0.4 μg/mL (1.1 μM) curcumin equivalent dose to SKOV-3, MCF-7, and OVCAR respectively. Notably, under the same conditions curcumin itself had an IC₅₀ of 7.8 μg/mL (21 μM) to SKOV-3, indicating that this curcumin-containing polymer has substantially greater cytotoxic activity than free curcumin. This is believed to be due to the fact that the pH sensitive polycurcumin of Example 8 is hydrophilic, water soluble (solubility >30 mg/mL) and stable in physiological conditions, whereas the free curcumin is almost insoluble in water, not stable at neutral conditions and has extremely low bioavailability. The polycurcumin of Example 8 remains longer in blood circulation and is readily taken up by cancer cells via diffusion or endocytosis cellular mechanisms. After the polycurcumin of Example 8 enters the cell, it gradually hydrolyzes in lower pH endosomes and lysosomes, discharging curcumin and bringing about curcumin accumulation inside cells, which results in higher bioavailability and enhanced cytotoxicity for the polycurcumin of Example 8, as compared to free curcumin.

The following example describes the results of testing to evaluate the in vivo antitumor activity of the polycurcumin of Example 8.

### Example 11

### In Vivo Antitumor Activity of Polycurcumin 8 to SKOV-3 Xenografts

The in vivo antitumor activity of the polycurcumin of Example 8 was further evaluated using SKOV-3 xenografts in an animal model. Athymic nude mice (BALB/c nu/nu, Charles River) were maintained in compliance with the policy on animal care expressed in the National Research Council guidelines (NRC 1985) and all experiments were approved and supervised by the Institutional Animal Care and Use Committee (IACUC) at the University of Wyoming. Mice were maintained in a pathogen-free environment under controlled temperature (24° C.) and lighting (12L:12D) conditions. Autoclaved rodent chow and sterilized water were supplied ad libitum.

SKOV-3 cells (5×10⁶ suspended in 2.0 mL PBS) were injected into the abdominal cavity of mature nude mice (12˜18 weeks). The mice were randomly divided into treatment group and control group (n=6) at five weeks post-inoculation (when tumors along the mesentery are well established). The treatment group was injected intravenously (i.v.) through the tail vain with polycurcumin 8 in 0.1 mL PBS based on a dose of 100 mg/kg and the control group was injected with 0.1 mL of PBS. The mice were sacrificed and dissected 48 h after the injection. All tumor tissues were collected and the total tumor weight of each mouse was measured. The difference in tumor weight between control group and treatment group was used as an as an overall mark of antitumor activity of the polycurcmin 8 against the SKOV-3 xenografts.

The athymic nude mice bearing the human SKOV-3 ovarian intraperitoneal tumor were treated with a single i.v. injection of the polycurcumin of Example 8 at a 100 mg/kg dose or PBS (control) through the mice tail veins. Assignments to treatments were made at random. Treatment comparisons were made by analysis of variance and protected least significant difference or Student's t-test. Contrasts were considered different at P<0.05. Data are presented as means±standard errors.

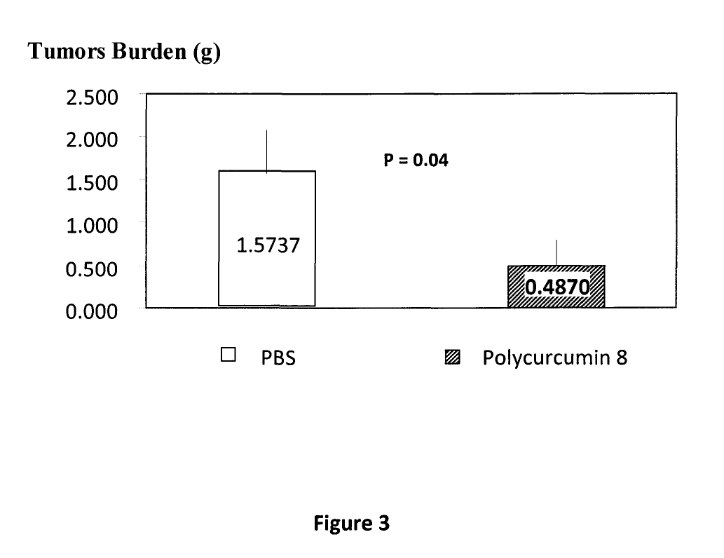

As shown in FIG. 3, significant antitumor activity was observed. The control group had an average tumor burden of 1.57 g while polymer-treated group had 0.49 g. The polymer decreased 68% tumor growth compared to control group, suggesting the remarkable tumor growth inhibition ability of the polycurcumin of is Example 8.

The following four examples relate to the preparation and assessment of biological activity of pharmaceutical dose forms that incorporate the curcumin-containing polymers and water-soluble curcumin derivatives of the present invention.

### Example 12

### Preparation of Vesicles from CurcuMin Modified with Tetraethyleneglycol Methyl Vinyl Ether

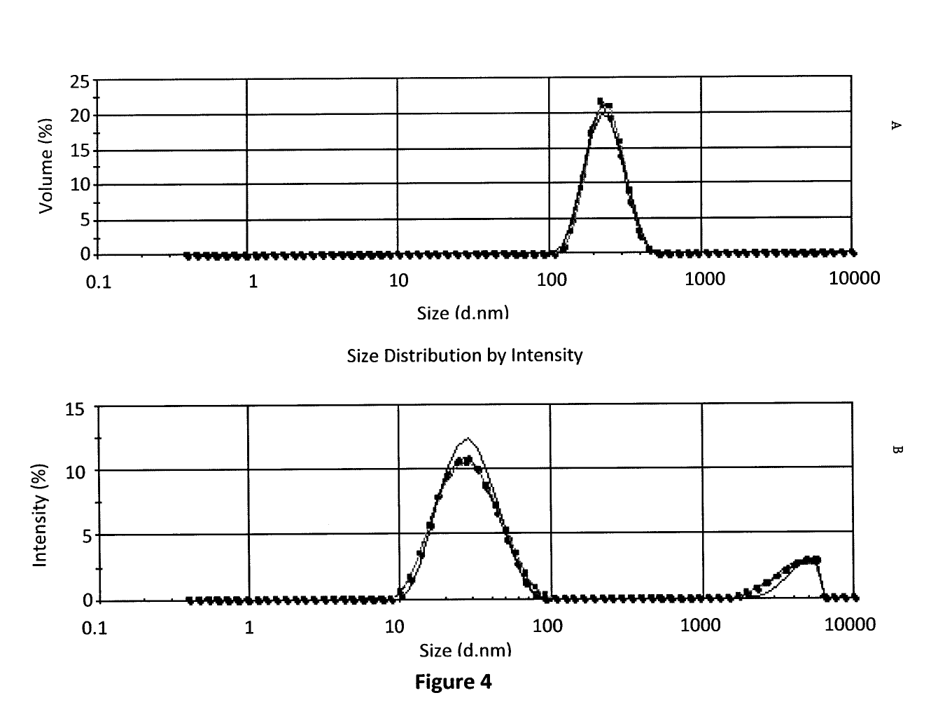

1.0 g curcumin, 3.0 g tetraethyleneglycol methyl vinyl ether, and 20 μg toluenesulfonic acid were dissolved in 40 mL anhydrous tetrahydrofuran. After the solution was stirred at room temperature for 10 hours, a large amount of anhydrous ether was added to precipitate the desired product. 0.1 g of this product was dissolved in 2 mL acetone and the acetone solution was added dropwise into 100 mL deionized water. After the solution was dialyzed against deionized water to remove acetone, the final vesicle solution was obtained with a size distribution as shown in FIG. 4a.

### Example 13

### Preparation of Nanoparticles from Curcumin Modified with Polyethylene Glycol Methyl Ether Acrylate

9.0 g of PEG methyl ether acrylate (Mn˜454), 2.3 g of 3-s mercaptopropionic acid and 0.1 mL of triethylamine were mixed in 100 mL anhydrous THF. After the mixture was stirred at room temperature for 24 hours, an excess of anhydrous ether was added to precipitate the PEG oligomer. 5.6 g of this oligomer, 2.2 g of DCC, 1.7 g of curcumin and 0.1 g of DMAP were dissolved in 50 mL of anhydrous THF. This solution was filtered to remove precipitate after stirring at room temperature for 24 hours and the filtrate was precipitated into an excess of anhydrous ether. The obtained product was further purified by reprecipitation from THF solution with anhydrous ether and dried under vacuum at room temperature. The nanoparticle solution was finally prepared by dissolving this product in deionized water or PBS solution, with a size distribution as shown in FIG. 4b.

### Example 14

### Preparation of Prodrug Carrier Vesicles from Curcumin with One Phenyl Hydroxyl Modified with Polyethylene Glycol Methyl Ether Acrylate

9.0 g of PEG methyl ether acrylate (Mn˜454), 2.3 g of 3-mercaptopropionic acid and 0.1 mL of triethylamine were mixed in 100 mL anhydrous THF. After the mixture was stirred at room temperature for 24 hours, an excess of anhydrous ether was added to precipitate the PEG oligomer. 2.8 g of this oligomer and 1.1 g of DCC were mixed in 30 mL anhydrous THF. This mixture was added dropwise into 30 mL THF solution containing 4.0 g of curcumin and 0.1 g of DMAP. After the THF solution was stirred at room temperature for 12 hours, the precipitate was removed by filtration and the filtrate was precipitated into an excess of anhydrous ether. The precipitate was further purified by reprecipitation two times from THF solution with anhydrous ether and dried under vacuum at room temperature to yield the final product. This product also formed nanoparticles when it was dissolved in water.

Both vesicles and nanoparticles formed from modified curcumin can be used as carriers for other prodrugs such as camptothecin, doxorubicin, cis-platin, paclitaxel, and etc. For example, by following steps, camptothecin (CPT) can be loaded into the vesicles/nanoparticles. 10 mg of CPT was dissolved in 2 ml DMSO and this DMSO solution was added dropwise into 50 mL deionized water containing 0.2 g of the above prepared product. After the solution was dialyzed against deionized water to remove DMSO, the CPT-loaded vesicles/nanoparticles was finally obtained, which were tested in a cytotoxicity assay as described below.

### Example 15

### MTT Assay of Curcumin Prodrugs

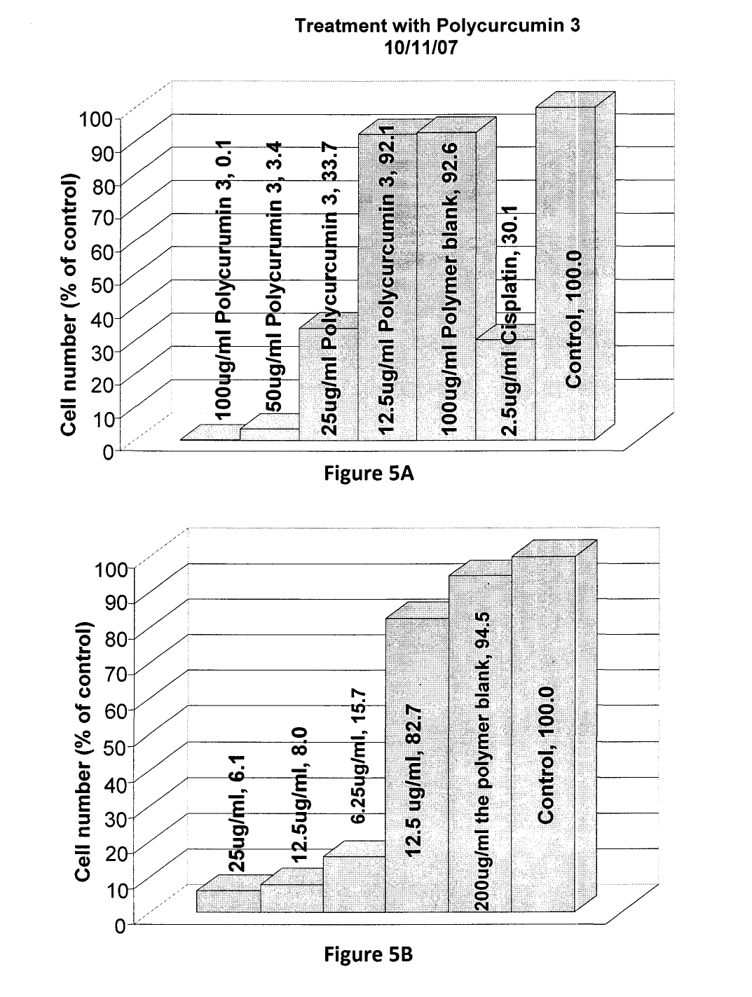

The cytotoxicity of curcumin prodrugs in the form of both a curcumin-containing polyester and certain curcumin derivatives of the invention was assessed by a standard MIT assay, using the SKOV ovarian cancer cells as targets. The treatment time for the MTT assay was 24 hours and post-treatment time was 72 hours. The results of such assays, using varying amounts of the curcumin-containing polymer of Example 1, above, and the curcumin derivative of Example 13, above, are shown in FIG. 5a and FIG. 5b, respectively. These results demonstrate that the curcumin-containing polymers and curcumin derivatives of this invention are effective for inducing cancer cell death or suppressing cell growth. The results shown by the bars in FIGS. 5a and 5b, which includes polymer blanks and controls (absence of agent), are the mean values for three experiments.

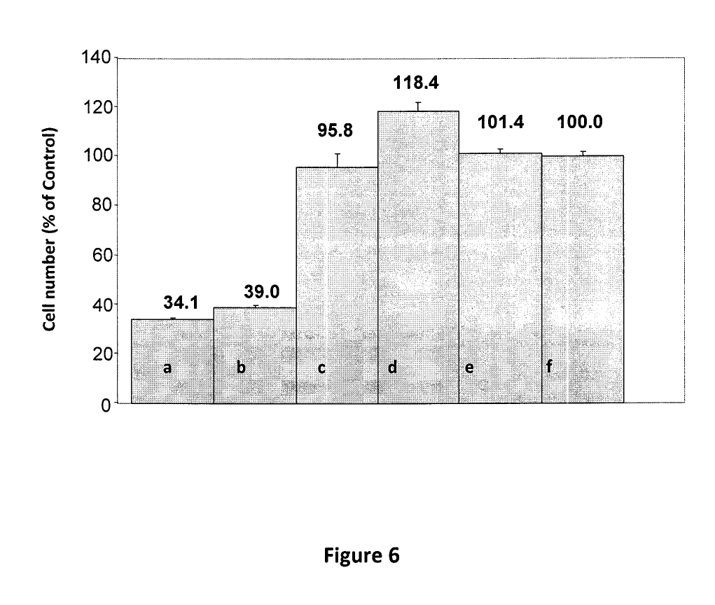

Additional cytotoxicity test results appear in FIG. 6. These results indicate that the CPT-loaded curcumin prodrug vesicles, prepared as described in Example 14, above, exhibit a much stronger cytotoxicity to cancer cells than either the vesicles or CPT alone. The results shown by the bars are the mean values for three experiments. The error bars indicate the standard deviation for each set of experiments.

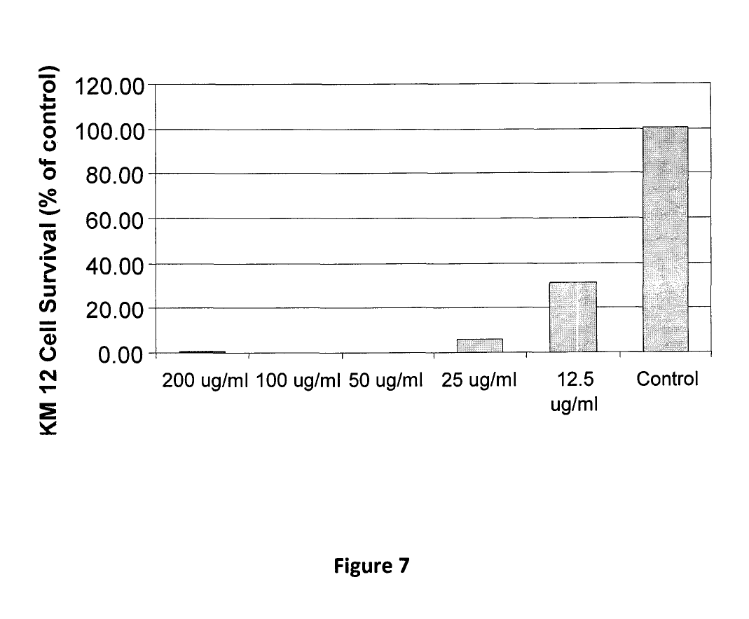

Similar cytotoxicty testing of the curcumin derivative of Example 13 was carried out using KM12 colon cancer cells. The results are set forth in FIG. 7.

The foregoing examples demonstrate that polycurcumin of Example 8 of this invention are hydrophilic, water soluble, and i.v. injectable. The polycurcumin of Example 8, in particular, was stable at physiological conditions and had very high curcumin loading content (21%) and loading efficiency (88%). MTT assay result showed that it is highly cytotoxic to SKOV-3, MCF-7, and OVCAR cancer cell lines with IC₅₀ of 1.2 μg/mL (3.3 μM), 1.4 μg/mL (3.8 μM) and 0.4 μg/mL (1.1 μM), respectively, based on curcumin equivalent dose. In vivo, the polymer showed remarkable antitumor activity in SKOV-3 i.p. tumor xenografts animal model.

A number of patent and non-patent publications are cited throughout the foregoing specification in order to describe the state of the art to which this invention pertains. The entire disclosure of each of these publications is incorporated by reference herein.

While certain embodiments of the present invention have been described and/or exemplified above, various other embodiments will be apparent to those skilled in the art from the foregoing specification. The present invention is, therefore, not limited to the particular embodiments described and/or exemplified, but is capable of considerable variation and modification without departure from is the scope of the appended claims. Furthermore, the transitional terms “comprising”, “consisting essentially of” and “consisting of', when used in the appended claims, in original and amended form, define the claim scope with respect to what unrecited additional claim elements or steps, if” any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All curcumin-containing polymers, water soluble curcumin derivatives and methods of use thereof that embody the present invention can, in alternate embodiments, be more specifically defined by any of the transitional terms “comprising”, “consisting essentially of” and “consisting of'.

### REFERENCES

1. Somparn, P.; Phisalaphong, C.; Nakornchai, S.; Unchern, S.; Morales Noppawan, P., Comparative antioxidant activities of” curcumin and its demethoxy and hydrogenated derivatives. Biol. Pharm. Bull. 2007, 30, (1), 74-8.
2. Menon Venugopal, P.; Sudheer Adluri, R., Antioxidant and anti-inflammatory properties of curcumin. Advances in experimental medicine and biology 2007, 595, 105-25.
3. Chen, D.; Nie, M.; Fan, M.-w.; Bian, Z., Anti-Inflammatory Activity of Curcumin in Macrophages Stimulated by Lipopolysaccharides from Porphyromonas gingivalis. Pharmacology 2008, 82, (4), 264-269.
4. Srivastava, R.; Dikshit, M.; Srimal, R. C.; Dhawan, B. N., Antithrombotic effect of curcumin Thromb. Res. 1985, 40 ((3)), 413-417.
5. Nguyen, K. T.; Su, S.-H.; Sheng, A.; Wawro, D.; Schwade, N., D.; Brouse, C. F.; Greilich, P. E.; Tang, L.; Eberhart, R. C., In vitro hemocompatibility studies of drug-loaded poly-(L-lactic acid) fibers Biomaterials 2003, 245, ((28)), 191-201
6. Shankeshi, J.; Aukunuru, J., Preparation, characterization and evaluation of antidiabetic activity of a liposomal formulation encapsulating curcumin, an ayurvedic natural product. Pharmacist (India) 2008, 3, (2), 1-5.
7. Kuhad, A.; Chopra, K., Curcumin attenuates diabetic encephalopathy in rats: Behavioral and biochemical evidences. Eur. J. Pharmacol. 2007, 576, (1-3), 34-42.
8. Mahalakshmi, K.; Prabhakar, J.; Sukumaran, V. G., Antibacterial activity of Triphala, GTP & curcumin on Enterococci faecalis. Biomedicine (India) 2006, 26, (3 & 4), 43-46.
9. De, R.; Kundu, P.; Swarnakar, S.; Ramamurthy, T.; Chowdhury, A.; Nair, G. B.; Mukhopadhyay, A. K., Antimicrobial activity of curcumin against Helicobacter pylori isolates from India and during infections in mice. Antimicrob. Agents Chemother. 2009, 53, (4), 1592-1597.
10. Kiso, Y.; Suzuki, Y.; Watanabe, N.; Oshima, Y.; Hikino, H., Validity of the oriental medicines. Part 53. Liver-protective drugs. Part 8. Antihepatotoxic principles of Curcuma longa rhizomes. Planta Med 1983, 49, (3), 185-7.
11. Mohamed, H. E.; Latif, A. A.; Thabet, S. M., Effect of curcumin on cadmium-induced hepatic toxicity in rats. J. Drug Res. 1998, 22, (1-2), 107-120.
12. Funk, J. L.; Timmermann, B. N., Translational investigation of turmeric for arthritis treatment: a review of lessons learned. Nat. Prod. Commun. 2006, 1, (11), 1061-1066.
13. Joe, B.; Lokesh, B. R., Prophylactic and therapeutic effects of n-3 polyunsaturated fatty acids, capsaicin, and curcumin on adjuvant induced arthritis in rats. J. Nutr. Biochem. 1997, 8, (7), 397-407.
14. Deodhar, S. D.; Sethi, R.; Srimal, R. C., Preliminary study on antirheumatic activity of curcumin (diferuloyl methane). Indian J. Med. Res. 1980, 71, 632-634.
15. Ringman, J. M.; Frautschy, S. A.; Cole, G. M.; Masterman, D. L.; Cummings, J. L., A potential role of the curry spice curcumin in Alzheimer's disease. Curr. Alzheimer Res. 2005, 2, (2), 131-136.
16. Balasubramanian, K., Molecular Orbital Basis for Yellow Curry Spice Curcumin's Prevention of Alzheimer's Disease. J. Agric. Food Chem. 2006, 54, (10), 3512-3520.
17. Baum, L.; Lam Christopher Wai, K.; Cheung Stanley, K.-K.; Kwok, T.; Lui, V.; Tsoh, J.; Lam, L.; Leung, V.; Hui, E.; Ng, C.; Woo, J.; Chiu Helen Fung, K.; Goggins William, B.; Zee Benny, C.-Y.; Cheng King, F.; Fong Carmen Yuet, S.; Wong, A.; Mok, H.; Chow Moses Sing, S.; Ho Ping, C.; Ip Siu, P.; Ho Chung, S.; Yu Xiong, W.; Lai Caroline Yau, L.; Chan, M.-H; Szeto, S.; Chan Iris Hiu, S.; Mok, V., Six-month randomized, placebo-controlled, double-blind, pilot clinical trial of curcumin in patients with Alzheimer disease. J. Clin. Psychopharmacol. 2008, 28, (1), 110-3.
18. Panjehshahin, M. R.; Owji, A. A.; Mehrabani, D.; Mirzaee, M.; Vasei, M., Effect of curcumin on cholesterol gallstone induction in rats. J. Appl. Anim. Res. 2003, 23, (1), 75-80.
19. Soni, K. B.; Kuttan, R., Effect of oral curcumin administration on serum peroxides and cholesterol levels in human volunteers. Indian J. Physiol. Pharmacol. 1992, 36, (4), 273-5.
20. Singh, S.; Aggarwa, B. B., Activation of Transcription Factor NF-kB Is Suppressed by Curcumin (Diferulolylmethane). J. Biol. Chem. 1995, 270, (42), 24995-25000.
21. Kunnumakkara, A. B.; Anand, P.; Aggarwal, B. B., Curcumin inhibits proliferation, invasion, angiogenesis and metastasis of different cancers through interaction with multiple cell signaling proteins. Cancer Lett. 2008, 269 (2), 199-225.
22. Farombi, E. O.; Shrotriy, S.; Surh, Y.-J., Kolaviron inhibits dimethyl nitrosamine-induced liver injury by suppressing COX-2 and iNOS expression via NF-κB and AP-1. Life Sciences 2009, 84, (149-155).
23. Aggarwal, B. B.; Surh, Y. H.; Shishodia, S., The molecular targets and therapeutics of curcumin in health and disease Springer: 2007; Vol. 596.
24. Liu, J. Y.; Lin, S. J.; Lin, J. K., Inhibitory effects of curcumin on protein kinase C activity induced by 12-O-tetradecanoylphorbol-13-acetate in NIH 3T3 cells. Carcinogenesis 1993, 14, 857-861.
25. Shukla, S.; Zaher, H.; Hartz, A.; Bauer, B.; Ware, J. A.; Ambudkar, S. V., Curcumin Inhibits the Activity of ABCG2/BCRP1, a Multidrug Resistance-Linked ABC Drug Transporter in Mice. Pharmaceutical Research 2008, 26, (2), 480-487.
26. Zhang, W.; Tan, T. M. C.; Lim, L.-Y., Impact of Curcumin-Induced Changes in P-Glycoprotein and CYP3A Expression on the Pharmacokinetics of Peroral Celiprolol and Midazolam in Rats. Drug Metabolism and Disposition 2006, 35, (1), 110-115.
27. Sandur, S. K.; Pandey, M. K.; Sung, B.; Ahn, K. S.; Murakami, A.; Sethi, G.; Limtrakul, P.; Badmaev, V.; Aggarwal, B. B., Curcumin, demethoxycurcumin, bisdemethoxycurcumin, tetrahydrocurcumin and turmerones differentially regulate anti-inflammatory and anti-proliferative responses through a ROS-independent mechanism. Carcinogenesis 2007, 28, (8), 1765-1773.
28. Song, G.; Mao, Y. B.; Cai, Q. F.; Yao, L. M.; Ouyang, G. L.; Bao, S. D., Curcumin induces human HT-29 colon adenocarcinoma cell apoptosis by activating p53 and regulating apoptosis-related protein expression. Braz. J. Med. Biol. Res. 2005, 38, (12), 1791-1798.
29. Li, L.; Braiteh, F. S.; Kurzrock, R., Liposome-encapsulated curcumin: in vitro and in vivo effects on proliferation, apoptosis, signaling, and angiogenesis. Cancer 2005, 104, 1322-1331.
30. Purkayastha, S.; Berlinerb, A.; Fernandoa, S. S.; Ranasinghe, B.; Ray, I.; Tariq, H.; Banerjee, P., Curcumin blocks brain tumor formation. Brain Research 2009, 1266, 130-138.
31. Cheng, A. L.; Hsu, C. H.; Lin, J. K.; Hsu, M. M.; Ho, Y. F.; Shen, T. S.; Ko, J. Y.; Lin, J. T.; Lin, B. R.; Ming-Shiang, W.; Yu, H. S.; Jee, S. H.; Chen, G. S.; Chen, T. M.; Chen, C. A.; Lai, M. K.; Pu, Y. S.; Pan, M. H.; Wang, Y. J.; Tsai, C. C.; Hsieh, C. Y., Phase I clinical trial of curcumin, a chemopreventive agent, in patients with high-risk or pre-malignant lesions Anticancer Res. 2001, 21 (4B), 2895-2900.
32. Sharma, R. A. E., S. A.; Platton, S. L.; Cooke, D. N.; Shafayat, A.; Hewitt, H. R.; Marczylo, T. H.; Morgan, B.; Hemingway, D.; Plummer, S. M.; Pirmohamed, M.; Gescher, A. J.; Steward, W. P., Phase I clinical trial of oral curcumin: biomarkers of systemic activity and compliance. Clin. Cancer Res. 2004, 10, 6847-6854.
33. Anand, P.; Kunnumakkara, A. B.; Newman, R. A.; Aggarwa, B. B., Bioavailability of Curcumin: Problems and Promises. Molecular Pharmaceutics 2007, 4, (6), 807-818.
34. Wang, Y.-J.; Pan, M.-H.; Cheng, A.-L.; Lin, L.-I.; Ho, Y.-S.; Hsieh, C.-Y.; Lin, J.-K., Stability of curcumin in buffer solutions and characterization of its degradation products. Journal of Pharmaceutical and Biomedical Analysis 1997, 15 1867-1876.
35. Huang, M.-T.; Newmark, H. L.; Frenkel, K., Inhibitory Effects of Curcumin on Tumorigenesis in Mice. Journal of Cellular Biochemistry Supplement 1997, 27, 26-34.
36. Chen, C.; Johnston Thomas, D.; Jeon, H.; Gedaly, R.; McHugh Patrick, P.; Burke Thomas, G.; Ranjan, D., An in vitro study of liposomal curcumin: stability, toxicity and biological activity in human lymphocytes and Epstein-Barr virus-transformed human B-cells. Int. J. Pharm. 2009, 366, (1-2), 133-9.
37. Batik, A.; Praveen, N.; Priyadarsini, K. I.; Kumar, M.; Mohan, H., Curcumin loaded hydrogels as probable drug delivery systems. Natl. Acad. Sci. Lett. (India) 2005, 28, (11 & 12), 383-388.
38. Vidyalakshmi, K. R., K. N.; Kumar, T. M.; Pramod, S., Studies on Formulation and In Vitro Evaluation of PVA/Chitosan Blend Films for Drug Delivery. Journal of Macromolecular Science, Pure and Applied Chemistry 2004, A41, (10), 1115-1122.
39. Suresh, S.; Prasad, K., Solid dispersions of curcumin. Indian J. Pharm. Sci. 1999, 61, (2), 131-133.
40. Paradkar, A.; Ambike, A. A.; Jadhav, B. K.; Mahadik, K. R., Characterization of curcumin-PVP solid dispersion obtained by spray drying. Int. J. Pharm. 2004, 271, (1-2), 281-286.
41. Bisht, S.; Feldmann, G.; Soni, S.; Ravi, R.; Karikar, C.; Maitra, A.; Maitra, A., Polymeric nanoparticle-encapsulated curcumin (“nanocurcumin”): a novel strategy for human cancer therapy. J. Nanobiotechnol. 2007, 5, (3), 1-18.
42. Sou, K. I., Shunsuke; Takeoka, Shinji; Tsuchida, Eishun., Loading of curcumin into macrophages using lipid-based nanoparticles. International Journal of Pharmaceutics 2008, 352, (1-2), 287-293.
43. Shi, W.; Dolai, S.; Rizk, S.; Hussain, A.; Tariq, H.; Averick, S.; L′Amoreaux, W.; Idrissi, A. E.; Banerjee, P.; Raja, K., Synthesis of Monofunctional Curcumin Derivatives, Clicked Curcumin Dimer, and a PAMAM Dendrimer Curcumin Conjugate for Therapeutic Applications Org Lett 2007, 9, (26), 5461-5464.
44. Safavy, A.; Raisch, K. P.; Mantena, S.; Sanford, L. L.; Sham, S. W.; Krishna, N. R.; Bonner, J. A., Design and Development of Water-Soluble Curcumin Conjugates as Potential Anticancer Agents. J. Med. Chem. 2007, 50, 6284-6288.
45. Meister, A.; Anderson, M. E., Glutathione. Ann. Rev. Biochem. 1983, 52, 711-760.
46. Kallinowski, F.; Vaupel, P., pH distributions in spontaneous and isotransplanted rat tumors. Br. J. Cancer 1988, 58, 314-321.
47. Engin, K.; Leeper, D. B.; Cater, J. R.; Thistlethwaite, A. J.; Tupchong, L.; McFarlane, J. D., Extracellular pH distribution in human tumours. Int. J. Hyperthermia 1995, 11, (2), 211-216.
48. Thistlethwaite, A. J.; Leeper, D. B.; Moylan, D. J. r.; Nerlinger, R. E., pH distribution in human tumors. Int. J. Rad. Oncol. Biol. Phys. 1985, 11, (9), 1647-1652.
49. Ireson, C.; OM S.; Jones, D. J. L.; Verschoyle, R.; Lim, C.-K.; Jin-Li Luo; Howells, L.; Plummer, S.; Jukes, R.; Williams, M.; Steward, W. P.; Gescher, A., Characterization of Metabolites of the Chemopreventive Agent Curcumin in Human and Rat Hepatocytes and in the Rat in Vivo, and Evaluation of Their Ability to Inhibit Phorbol Ester-induced Prostaglandin E2 Production. Cancer Research 2001, 61, (1058-1064).
50. Kunwar, A.; Barik, A.; Mishra, B.; Rathinasamy, K.; Pandey, R.; Priyadarsini, K. I., Quantitative cellular uptake, localization and cytotoxicity of curcumin in normal and tumor cells. Biochimica et Biophysica Acta 2008, 1780, 673-679.
51. Lin, S.-S.; Huang, H.-P.; Yang, J.-S.; Wud, J.-Y.; Hsai, T.-C.; Lin, C.-C.; Lin, C.-W.; Kuo, C.-L.; Wood, W. G.; Chung, J.-G., DNA damage and endoplasmic reticulum stress mediated curcumin-induced cell cycle arrest and apoptosis in human lung carcinoma A-549 cells through the activation caspases cascade- and mitochondrial-dependent pathway. Cancer Lett. 2008, 272, (1), 77-90.
52. Choudhuri, P. S. T.; Das, T.; Sa, G., Curcumin selectively induces apoptosis in deregulated cyclin D1-expressed cells at G2 phase of cell cycle in a p53-dependent manner. J. Biol. Chem. 2005, 20, 20059-20068.
53. Jacot, J. G.; Wong, J. Y., Endothelial Injury Induces Vascular Smooth Muscle Cell Proliferation in Highly Localized Regions of a Direct Contact Co-culture System. Cell Biochem Biophys 2008, 52, 37-46.
54. Perkin, J. Chem. Soc. (Trans.), 85: 64 (1904).
55. German Patent 859145
56. Ireson, Can. Res., 61: 1058-64 (2001)
57. U.S. Pat. No. 4,938,949
58. Harrison's Principles of International Medicine, 13^{th} ed., 1814-82 (1996)
59. Lowe et al., Cell, 74: 957-967 (1993)
60. U.S. Pat. No. 5,821,072

1. A polymer comprising curcumin as a constituent monomer.

2. A polymer having a backbone of repeating structural units, said repeating structural units being the same or different, at least one of said repeating structural units comprising a curcumin monomer residue.

3. The polymer according to claim 2, wherein said repeating structural units are the same.

4. The polymer according to claim 2, wherein said curcumin monomer residue is chemically bound to at least one other monomer residue and said repeating structural units comprise polyester units.

5. The polymer according to claim 4, wherein said at least one other monomer residue is a residue of a polycarboxylic acid, polycarboxylic acid anhydries or polycarboxylic acid halide.

6. The polymer according to claim 4, wherein said at least one other monomer residue is a residue of oxalic acid, succinic acid, 3,3′-dithiodipropronic acid, terephthalic acid, benzophenone-3,3′,4,4′-tetracarboxylic dianhydride, cyclobutane-1,2,3,4-tetracarboxylic dianhydride, tetrahydrofuran-2,3,4,5-tetracarboxylic anhydride, diethylenetriaminepentacetic acid anhydride, pyromellitic dianhydride.

7. The polymer according to claim 2, wherein said curcumin monomer residue is chemically bound to at least one other monomer residue and said repeating structural units comprise polyether units.

8. The polymer according to claim 7, wherein said at least one other monomer residue is a divinyl compound.

9. The polymer according to claim 7, wherein said at least one other monomer is a residue of divinyl sulfone, divinyl sulfoxide, 1,4-butanediol divinyl ether, 1,4-cyclohexanedimethanol divinyl ether, 1,4-divinyl-1,1,2,2,3,3,4,4-octamethyltetrasilane, bis[4-(vinyloxy)butyl]adipate, tri(ethylene glycol) divinyl ether, di(ethylene glycol) divinyl ether and polyethylene glycol divinyl ether.

10. The polymer according to claim 2, wherein said repeating structural units are different and said polymer backbone further includes a repeating structural unit comprising a polyether glycol residue.

11. The polymer according to claim 10, wherein said polyether glycol residue is a residue of polyethyleneglycol (PEG), polypropyleneglycol (PPG), and polyethyleneglycol-polypropylene glycol block copolymers.

12. The polymer according to claim 10, wherein said polyether glycol residue is a polyethylene glycol residue.

13. The polymer according to claim 12, wherein said polyethylene glycol residue is a residue of a polyethylene glycol having a molecular weight in the range of 200 to 20,000.

14. A polymer according to claim 1, wherein curcumin is copolymerized with at least one monomer from the group of polycarboxylic acids, polycarboxylic acid anhydrides, divinyl compounds, polycarboxylic acid halides, and polyetherglycol monomers.

15. A polymer according to claim 14, comprising a polymer backbone of curcumin and a comonomer from the group of polycarboxylic acids, polycarboxylic acid anhydrides, divinyl compounds, polycarboxylic acid halides, and polyetherglycols, and a moiety effective for adjusting the water solubility of said copolymer, chemically bound to said backbone.

16. A polymer according to claim 15, wherein said moiety is a polyetherglycol, polyvinylpyrrolidone (PVP), polyvinyl alcohol (PVA), polyethyleneimine (PEI) and poly[N-(2-hydroxypropyl)methacrylamide] (PHPMA), or polyglutamic acid moiety.

17. A polymer according to claim 15, wherein said moiety is at leaset one polyetherglycol moiety selected from the group of a polyethyleneglycol, polypropyleneglycol, and polyethyleneglycol-polypropyleneglycol block copolymer.

18. A polymer according to claim 14, comprising a polymer backbone of comonomers selected from the group of polycarboxylic acids, polycarboxylic acid anhydrides, divinyl compounds, polycarboxylic acid halides, and polyetherglycols and a curcumin moiety chemically bound to said backbone.

19. A pharmaceutical preparation comprising a polymer of claim 1 and a carrier medium.

20. Colloidal particles comprising the polymer of claim 1.

21. A pharmaceutical preparation comprising colloidal particles of claim 20.

22. A curcumin derivative of the formula: 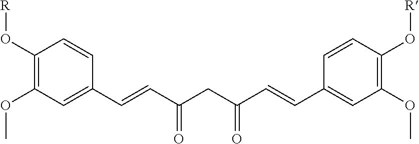
wherein one or both of R and R′ represent a water-soluble moiety, and when only one of R and R′ represent a water-soluble moiety, the other represents hydrogen.

23. The curcumin derivative of claim 22, wherein said water-soluble moiety imparts an amphiphilic character to said derivative.

24. The curcumin derivative of claim 22, wherein each of R and R′ represent a water-soluble moiety selected from tetraethyleneglycol methyl vinyl ether, polyethyleneglycol methyl vinyl ether, polyethyleneglycol, polyvinylpyrrolidone, polyvinyl alcohol, polyethyleneimine and poly[N-(2-hydroxypropyl)methacrylamide](PHPMA), polyglutamic acid moieties, each said moiety having a molecular weight in the range of 100-200,000.

25. A pharmaceutical preparation comprising a curcumin derivative of claim 22 and a carrier medium.

26. Colloidal particles comprising a curcumin derivative of claim 22.

27. The colloidal particles of claim 26 which are in the form of vesicles.

28. The colloidal particles of claim 26 which are in the form of nanoparticles.

29. A pharmaceutical preparation comprising the colloidal particles of claim 27.

30. The pharmaceutical preparation of claim 29 further comprising an anti-neoplastic agent.

31. The pharmaceutical preparation of claim 30, wherein said anti-neoplastic agent is selected from the group of camptothecin, doxorubicin, aclarubicin, bleomycin, peplomycin, chromomycin, cis-platin, paclitaxel, vincristin, colchioinamide, curcumin.

32. A method for the treatment of a proliferative disease, in a patient in need of said treatment, comprising administering to said patient an effective amount of a pharmaceutical preparation of claim 1.

33. The method of claim 32, wherein said pharmaceutical preparation administered for the treatment of cancer.

34. A pharmaceutical preparation comprising the colloidal particles of claim 28.
